(19) United States
(12) Patent Application Publication
(10) Pub. No.: US 2024/0296918 A1
(43) Pub. Date: Sep. 5, 2024

(54) Joint Generation of a Molecular Graph and Three-Dimensional Geometry

(71) Applicant: D.E. Shaw research, LLC, New York, NY (US)

(72) Inventors: James Peter Roney, Santa Barbara, CA (US); Pavlos Maragkakis, New York, NY (US); Peter Skopp, Westport, CT (US)

(21) Appl. No.: 18/693,221

(22) PCT Filed: Sep. 28, 2022
(86) PCT No.: PCT/US2022/045016

§ 371 Date: Mar. 19, 2024

Related U.S. Application Data
(60) Provisional application No. 63/249,162, filed on Sep. 28, 2021.

(51) Int. Cl. G16C 20/50; G16C 20/70

(52) U.S. Cl. CPC G16C 20/50; G16C 20/70

(57) ABSTRACT

A machine-learning approach jointly generates molecular graphs and corresponding three-dimensional geometries, for example, for searching a chemical space of potential molecules with desired chemical properties. In some examples, molecules are generated incrementally by repeatedly adding atoms to a molecular graph as well as determining geometric (e.g., location) information for the added atoms until a complete molecule is generated. This incremental process can be stochastic enabling random sampling from a chemical space.

### CROSS-REFERENCES TO RELATED APPLICATIONS

This application claims the benefit of U.S. Provisional Application No. 63/249,162 filed on Sep. 28, 2021, which is incorporated herein by reference.

### BACKGROUND OF THE INVENTION

This document relates to joint generation of a molecular graph and three-dimensional geometry.

The extremely large size of chemical space prohibits exhaustive experimental and computational screens for molecules with desirable properties. This intractability has motivated the development of machine learning models that can propose novel molecules possessing specific characteristics. To date, most machine learning models have focused on generating molecular graphs (which describe the topology of covalently bonded atoms in a molecule), but do not generate three-dimensional (3D) coordinates for the atoms in these graphs. Unfortunately, such models are capable of generating geometrically implausible molecular graphs and cannot directly incorporate information about 3D geometry when optimizing molecular properties. It is also possible to build machine learning models that generate 3D coordinates without generating corresponding molecular graphs, but the lack of graphs makes certain downstream applications more challenging.

### SUMMARY OF THE INVENTION

In one aspect, in general, a molecular graph for a molecule and corresponding three-dimensional geometry for the molecule is generated incrementally. For at least some increments, a molecular graph and corresponding three-dimensional geometry for a partial molecule is extended by adding to the molecular graph as well as adding geometric information for atoms added in the increment to the molecular graph. A random set (“ensemble”) of such representations of molecules, each with a corresponding molecular graph and three-dimensional geometry, can be generated to match a distribution of desired molecules (e.g., having a desired chemical property).

Aspects have technical advantages in one or more ways. First, from a computational efficiency point of view, valid molecules (i.e., molecules that may be physically synthesized and/or may physically exist) may be generated with fewer computational resources (e.g., number of instructions and/or numerical computations executed per generated molecule) than with prior approaches to generating molecule candidates of similar quality. Second, the molecules generated by the approaches may have much higher rates of chemical validity, and/or much better atom-distance distributions, than those generated with previous models. This can result in fewer physical (i.e., experimental) and/or computational resources that are required to be expended for further screening of the molecule proposed by these approaches. Finally, these approaches have been found to advance the state of the art in geometric accuracy for generated molecules.

In this document, a “molecular graph” should be understood to be a representation of a molecule (or partial molecule) that encodes atoms and bonding information between the atoms but does not explicitly encode absolute or relative location information between the atoms. Conversely, “geometric information” should be understood to be a representation that explicitly encodes absolute or relative locations of atoms in a molecule, but does not explicitly encode connection information between atoms, such as the presence or type of bonds between atoms of the molecule.

Aspects may include one or more of the following features alone or in combinations.

The generated molecule is provided for further physical or simulated evaluation of its chemical properties.

The method for generating the molecule is adapted to preferentially generate molecules with a desired chemical property.

The desired chemical property can include having a low-energy geometry.

In at least some examples, a single atom is added in an increment, for example, with a completed molecule being generated by incrementally adding one atom at a time.

The extension of the molecular graph includes determining a label for each atom added in the increment and determining bonding information between each atom added and atoms of the partial molecule to which the increment is added.

The label for an atom identifies the element of the atom.

The bonding information include whether or not a bond is present and/or a bond type between the two molecules.

The adding of geometric information includes adding location information for each atom added in the increment.

Adding the location information includes at least one of (a) determining physical distance information of an atom in the increment to one or more atoms in the partial molecule, (b) determining physical angle information of an atom in the increment to two or more atoms in the partial molecule, and (c) determining both the physical distance information and the physical angle information.

The extension of the molecular graph depends at least in part on geometry of the partial molecule that is extended.

The molecule is formed in a random manner. For example, multiple molecules are formed with each molecule being randomly formed using a randomized procedure.

Forming a molecule using a randomize procedure includes determining a distribution (e.g., a probability distribution) over possible increments to the molecular graph, and selecting a particular increment in a random manner.

Determining the label for an atom added in the increment includes using a first artificial neural network that takes as input a representation of at least one of (a) a representation of the molecular graph of the partial molecule, (b) a representation of the three-dimensional geometry of the partial molecule, and (c) representations of both the molecular graph and the three-dimensional geometry of the partial molecule.

The output of the first artificial neural network includes a distribution of possible labels of the atom that is added.

Determining the bonding information for an atom added in the increment includes using a second artificial neural network that takes as input a representation of at least one of (a) a representation of the molecular graph of the partial molecule, (b) a representation of the three-dimensional geometry of the partial molecule, (c) a representation of the label or distribution of labels for an atom that is to be added, and (d) any combination of (a)-(c). Determining physical distance information of an atom in the increment to one or more atoms in the partial molecule includes using a third artificial neural network that takes as input at least (a) a representation of the three-dimensional geometry of the partial molecule, (b) a representation of a label or a distribution of labels of the atom to be added, (c) a representation of the molecular graph of the partial molecule, and (d) any combination of (a)-(c).

The third artificial neural network is used repeatedly to determine physical distance information to different atoms of the partial molecule.

Determining physical angle information of an atom in the increment to one or more atoms in the partial molecule includes using a fourth artificial neural network that takes as input at least (a) a representation of the three-dimensional geometry of the partial molecule, (b) a representation of a label or a distribution of labels of the atom to be added, (c) a representation of the molecular graph of the partial molecule, and (d) any combination of (a)-(c).

One or more of the first through fourth neural networks are trained using a molecular graph and three-dimensional geometry information for a database of valid molecules.

One or more of the first through fourth neural networks are trained using a molecular graph and three-dimensional geometry information for a database of molecules having a desired chemical property.

One or more of the first through fourth neural networks are adapted using a molecular graph and three-dimensional geometry information for a database of molecules having a desired chemical property after training them using a database of molecules that do not necessarily have the desired chemical property.

In an aspect, the invention provides a computer-implemented method for determining a data representation of a molecule. Especially, the method may comprise joint generation of a molecular graph and three-dimensional geometry for a molecule. In embodiments, the joint generation may include determining a data representation of an (initial) partial molecule. The joint generation may in embodiments further include repeating incremental modification of the partial molecule, such as in each repetition or such as in at least some of the repetitions, incrementally adding an increment comprising one or more atoms to the partial molecule. The repeating incremental modification of the partial molecule may, in embodiments, further include forming a data representation for the partial molecule to include a molecular graph including the one or more atoms and the geometric information for said one or more atoms. Yet further, in embodiments, the joint generation may include providing a final data representation of the partial molecule as a representation of the generated molecule.

In embodiments, incrementally adding the increment may include selecting the one or more atoms based on the partial molecule.

In embodiments, incrementally adding the increment may include selecting the one or more atoms based on the partial molecule.

Further, in embodiments, incrementally adding the increment may include adding the one or more atoms to the molecular graph of the partial molecule. Further, in embodiments, incrementally adding the increment may include determining the geometric information for the one or more atoms added in the increment to the molecular graph.

In embodiments, at least one of the incrementally adding of the increment comprising one or more atoms to the partial molecule, the selecting of the one or more atoms based on the partial molecule, the adding of the one or more atoms to the molecular graph of the partial molecule, and the determining of the geometric information for the one or more atoms may be performed using a machine learning model trained from a training set of molecules. Hence, in further embodiments, the incrementally adding of the increment comprising one or more atoms to the partial molecule may be performed using a machine learning model trained from a training set of molecules. Hence, in further embodiments, the selecting of the one or more atoms based on the partial molecule may be performed using a machine learning model trained from a training set of molecules. Hence, in further embodiments, the adding of the one or more atoms to the molecular graph of the partial molecule may be performed using a machine learning model trained from a training set of molecules. Hence, in further embodiments, the determining of the geometric information for the one or more atoms may be performed using a machine learning model trained from a training set of molecules.

In further embodiments, the machine learning model may comprise an artificial neural network.

In further embodiments, the training set of molecules may be selected according to desired properties of the generated molecule, such as desired chemical properties of the generated molecule.

Especially, the method may further comprise training the machine learning model from the training set of molecules.

Furthermore, in embodiments, the method may further comprise adapting the method to preferentially generate molecules with a desired chemical property. In particular, in embodiments, the method may comprise preferentially generating a molecule with a desired chemical property.

In further embodiments, the desired chemical property may include having a low-energy geometry.

In embodiments, the initial partial molecule may consist of a single atom.

In embodiments, in at least some iterations (also referred to as repetitions), a single atom may be added in an increment.

In further embodiments, in each iteration only a single atom may be added.

In embodiments, each may further include determining a label for each atom added in the increment, and determining bonding information between each atom added and (each) atom(s) of the partial molecule to which the increment is added.

In embodiments, the label for an atom may identify the element of the atom.

In embodiments, the bonding information may include at least one of an indication of whether or not a bond is present and a bond type between two molecules, such as whether or not a bond is present between two molecules, or such as a bond type between two molecules.

Further, in embodiments, the adding of geometric information may include adding location information for each atom added in the increment.

In further embodiments, adding the location information may include at least one of (a) determining physical distance information of an atom in the increment to one or more atoms in the partial molecule, (b) determining physical angle information of an atom in the increment to two or more atoms in the partial molecule, and (c) determining both the physical distance information and the physical angle information. Especially, in embodiments, adding the location information may include determining physical distance information of an atom in the increment to one or more atoms in the partial molecule. In further embodiments, adding the location information may include determining physical angle information of an atom in the increment to one or more atoms in the partial molecule. In further embodiments, adding the location information may include determining both the physical distance information and the physical angle information.

In embodiments, the incremental addition may depend at least in part on geometry of the partial molecule. In particular, in embodiments, the increment may depend at least in part on geometry of the partial molecule.

In embodiments, the molecular graph and the three-dimensional geometry may in embodiments be formed in a random manner.

In further embodiments, multiple molecules may be formed with each molecule being randomly formed using a randomized procedure.

In further embodiments, forming a molecule using the randomized procedure may include determining a distribution over possible increments to the molecular graph, and especially selecting a particular increment (from the possible increments) in a random manner.

In embodiments, determining the label for an atom added in the increment may include using a first artificial neural network that takes as input a representation of at least one of (a) a representation of the molecular graph of the partial molecule, (b) a representation of the three-dimensional geometry of the partial molecule, and (c) representations of both the molecular graph and the three-dimensional geometry of the partial molecule. In further embodiments, the first artificial neural network takes as input (a representation of) a representation of the molecular graph of the partial molecule. In further embodiments, the first artificial neural network takes as input (a representation of) a representation of the three-dimensional geometry of the partial molecule. In further embodiments, the first artificial neural network takes as input both (representations of) a representation of the molecular graph of the partial molecule and a representation of the three-dimensional geometry of the partial molecule.

Further, the output of the first artificial neural network may include a distribution of possible labels of the atom that is added.

In embodiments, determining the bonding information for an atom added in the increment may include using a second artificial neural network that takes as input (a representation of) at least one of (a) a representation of the molecular graph of the partial molecule, (b) a representation of the three-dimensional geometry of the partial molecule, and (c) a representation of the label or distribution of labels for an atom added. In further embodiments, the second artificial neural network takes as input (a representation of) a representation of the molecular graph of the partial molecule. In further embodiments, the second artificial neural network takes as input (a representation of) a representation of the three-dimensional geometry of the partial molecule. In further embodiments, the second artificial neural network takes as input (a representation of) a representation of the label or distribution of labels for an atom added.

In embodiments, determining physical distance information of an atom in the increment to one or more atoms in the partial molecule may include using a third artificial neural network that takes as input at least (a) a representation of the three-dimensional geometry of the partial molecule, (b) a representation of a label or a distribution of labels of the atom to be added, and (c) a representation of the molecular graph of the partial molecule. In further embodiments, the third artificial neural network may take as input a representation of the three-dimensional geometry of the partial molecule. In further embodiments, the third artificial neural network may take as input a representation of a label or a distribution of labels of the atom to be added. In further embodiments, the third artificial neural network may take as input a representation of the molecular graph of the partial molecule.

In embodiments, the third artificial neural network may be used repeatedly to determine physical distance information to different atoms of the partial molecule.

Further, in embodiments, determining physical angle information of an atom in the increment to one or more atoms in the partial molecule includes using a fourth artificial neural network that may take as input at least (a) a representation of the three-dimensional geometry of the partial molecule, (b) a representation of a label or a distribution of labels of the atom to be added, and (c) a representation of the molecular graph of the partial molecule. In further embodiments, the fourth artificial neural network may take as input a representation of the three-dimensional geometry of the partial molecule. In further embodiments, the fourth artificial neural network may take as input a representation of a label or a distribution of labels of the atom to be added. In further embodiments, the fourth artificial neural network may take as input a representation of the molecular graph of the partial molecule.

Yet further, in embodiments, one or more of the first through fourth neural networks, especially the first neural network, or especially the second neural network, or especially the third neural network, or especially the fourth neural network, may be trained using a molecular graph and three-dimensional geometry information for a database of valid molecules.

In further embodiments, one or more of the first through fourth neural networks especially the first neural network, or especially the second neural network, or especially the third neural network, or especially the fourth neural network, may be trained using a molecular graph and three-dimensional geometry information for a database of molecules having a desired chemical property.

In further embodiments, one or more of the first through fourth neural networks especially the first neural network, or especially the second neural network, or especially the third neural network, or especially the fourth neural network, may in embodiments be adapted using a molecular graph and three-dimensional geometry information for a database of molecules having a desired chemical property after training them using a database of molecules that do not necessarily have the desired chemical property.

In a further aspect, the invention may provide a non-transitory machine-readable medium comprising instructions stored thereon, said instructions when executed using a computer processor cause said processor to perform (all the steps of) the (computer-implemented) method of the invention.

In a further aspect, the invention may provide a non-transitory machine-readable medium comprising a representation of one or more trained machine learning models, said machine learning models imparting functionality to a system for generating molecules according to (the steps of) the (computer-implemented) method of the invention.

Hence, the invention may provide a computer-readable (storage) medium comprising instructions which, when executed by a computer, cause the computer to carry out (the steps of) the (computer-implemented) method of the invention.

In a further aspect, the invention may provide a data processing system comprising means for carrying out (the steps of) the (computer-implemented) method of the invention.

In a further aspect, the invention may provide a computer program product comprising instructions which, when the program is executed by a computer, cause the computer to carry out (the steps of) the (computer-implemented) method of the invention.

Other aspects and features as well as advantages will be understood form the entire content of this document.

### BRIEF DESCRIPTION OF THE DRAWINGS

FIG. 1 is flowchart illustrating a procedure to add an n+1^{st} increment to a partial molecule;

FIG. 2 is an illustration of an exemplary use of the procedure of FIG. 1;

FIG. 3 is a set of renderings of three-dimensional molecules produced when trained on various training sets listed on the left;

FIG. 4 is a set of renderings of three-dimension molecules produced with training for GEOM-QM9 in which the right column contains reference geometries, and the left two columns show the nearest neighbor to the reference geometries among the geometries generated by RDKit and the present GEN3D system;

FIG. 5 is a histograms of inter-atom distances for generated molecules and QM9 molecules with 19 total atoms;

FIG. 6 is a plot showing probability densities of ROCS scores for molecular graphs and geometries generated by GEN3D-gd (left-most peak), molecular graphs generated by GEN3D-ft with OpenEye Omega geometries (center peak), and molecular graphs and geometries generated by GEN3D-ft (right-most peak); and

FIG. 7 is a scatter plot of the similarity to the fine-tuning dataset of the molecules with the top 2% of ROCS scores for GEN3D-gd (cluster at lower left) and GEN3D-ft (cluster at upper-right).

### DETAILED DESCRIPTION

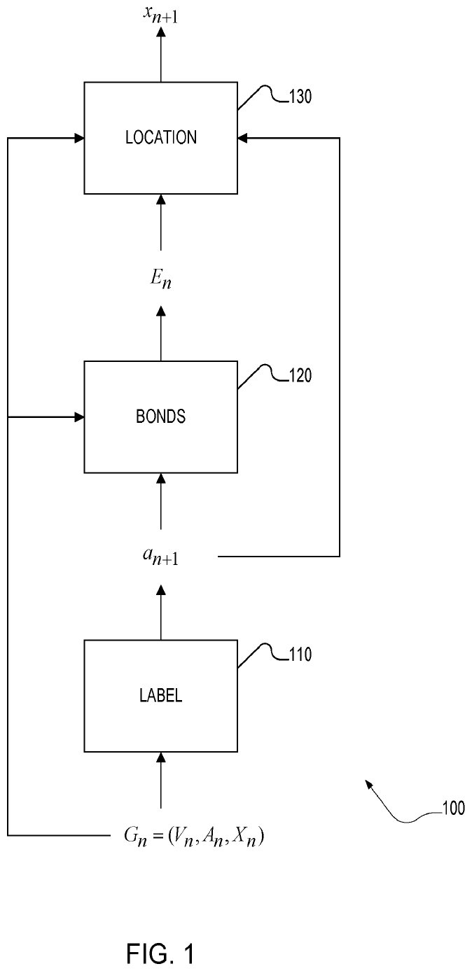

In one embodiment, an incremental procedure (which may also be referred to as an “iterative procedure”) is used to construct a data representation of a physical molecule by repeatedly adding to a partial molecule. Referring to FIG. 1, the process 100 for one repetitions (which may be referred to as an “iteration”) of the procedure for transforming an n^{th} data representation of a partial molecule to form the n+1^{st} data representation of a partial molecule (or a completed molecule) involves a succession of three steps. The n^{th} partial molecule is represented in a data structure Gₙ, that has label information, Vₙ, for the atoms of the partial molecule, bond information, Aₙ, for those atoms, and geometry information, Xₙ, for those atoms. The combination of Vₙ and Aₙ represent the molecular graph of the partial molecule, while Gₙ further incorporates geometric information. In a first step 110, a label, aₙ₊₁, for the next atom (or alternatively a complex of multiple atoms) to be added to the molecule is determined using a first process trained on one or more training molecular datasets (e.g., a “machine learning” process), in this embodiment implemented using an artificial neural network (ANN). For example, a probability distribution of possible labels is output from the process, and one label is selected at random from that distribution, or the randomly drawn label is determined directly without an explicit representation of the distribution of possible labels (e.g., using a generative neural network).

In a second step 120, the determined label in combination with the information representing the partial molecule are used to determine bonding information, Eₙ, which represents the presence of any bonds and their types between new atom, aₙ₊₁, and the atoms of the n^{th} partial molecule. This step preferably also uses a process trained on one or more training molecular datasets (e.g., a “machine learning” process), in this embodiment implemented using an artificial neural network (ANN).

Together, aₙ₊₁ and Eₙ represent the increment to be added to the molecular graph, without yet representing the geometric relationship of the new atom(s) of the increment relative to the n^{th} partial molecule.

In a third step 130, geometric coordinates (i.e., values specifying location information), xₙ₊₁ of the added atom(s) of the increment are determined based on the information of the incrementally updated molecular graph as well as the previously determined locations of the atoms of the partial molecule. In a preferred approach, this third step includes determining distances between the new atom(s) and coordinates of one or more atoms of the atoms of the partial molecule, as well as determining angles between the new atom(s) and atoms of the partial molecule. The distances and angles are combined to determine the geometric coordinates of the new atom. This step preferably also uses a process trained on one or more training molecular datasets (e.g., a “machine learning” process), in this embodiment implemented using an artificial neural network (ANN)

The computed label, aₙ₊₁, bond information, Eₙ, and coordinates, xₙ₊₁, are combined with Gₙ to form Gₙ₊₁, which is then used in the next repetition of the procedure.

The iterative procedure is completed when the label aₙ₊₁ is a “termination” label indicating that a complete molecule has been generated.

This randomized procedure can be repeated to form an ensemble of generated molecules.

In particular, the invention may provide a computer-implemented method for determining a data representation of a (generated) molecule, the method comprising joint generation of a molecular graph and three-dimensional geometry for a molecule. The joint generation may include determining a data representation of an initial partial molecule, i.e., a 1^{st} data representation of a partial molecule. The joint generation may further comprise transforming an n^{th} data representation of a partial molecule to form an n+1^{st} data representation of a partial molecule (or a completed molecule). The transforming of an n^{th} data representation of a (n^{th}) partial molecule to form an n+1^{st} data representation of a (n+1^{st}) partial molecule may herein be referred to as a “repetition”. The joint generation may especially comprise a plurality of repetitions, such as from a 1st data representation of the (initial) partial molecule to a 2^{nd} data representation of the (2^{nd}) partial molecule, and such as from the 2^{nd} data representation on to a 3^{rd} data representation, et cetera. In embodiments, the repeating incremental modification of the partial molecule, especially in each repetition, or especially in at least some of the repetitions, may comprise incrementally adding an increment comprising one or more atoms to the partial molecule, and forming a data representation for the partial molecule (including the increment) to include a molecular graph including the one or more atoms and the geometric information for said one or more atoms. Following the repeating incremental modification, especially the plurality of repetitions, the method may comprise providing a final data representation of the partial molecule as a representation of the (generated) molecule.

In some use cases, the iteration begins with an “empty” partial molecule. In other examples, the iteration begins with a partial molecule that has been constructed in another manner, for example, by selecting a part of a known molecule.

By virtue of the random selection of labels aₙ, an ensemble of molecules may be generated, for example, by repeating the entire process, or branching or backtracking during the generation process.

The one or more molecules generated in this manner are then available to be further evaluated, for example, with further physical synthesis and physical evaluation, or simulation and/or computational evaluation of its chemical properties. For example, simulation using approaches described in one of more of the following may be used: U.S. Pat. Nos. 7,707,016; 7,526,415; and 8,126,956; and PCT application PCT/US2022/020915, which are incorporated herein by reference.

As introduced above, a machine learning approach may be used for one or more of the steps illustrated in FIG. 1. A variety of model training approaches may be used. For example,
1. Train the model with some unbiased dataset of drug like molecules.
2. Take a modest size dataset (possibly the same as used in step 1) and run a computational screening tool against those to generate a rank order of predicted value, affinity, energy, and/or score. For example, this can be a docking score.
3. Take the top N molecules from the sorted list in step 2, and continue to train the existing trained network for a number of epochs with the new data (e.g., for fewer epochs than step 1).
4. Generated molecules from the network now should perform better on the docking to a target than the original model from step 1 that was generating random molecules.

As introduced above, at a high level, the system generates 3D molecules by adding atoms to a partially complete molecular graph, attaching them to the graph with new edges, and localizing them in 3D space. These approaches may be referred to as “GEN3D” in the discussion and figures below. One architecture for such a system consists of four 3D graph neural networks: an atom network (denoted F_{A}, and referred to as the “first artificial neural network”) for use in step 110 (shown in FIG. 1), an edge network (denoted F_{E} and referred to as the “second artificial neural network”) for use in step 120, and a distance network (denoted F_{D} and referred to as the “third artificial neural network”) and an angle network (denoted F_{θ} and referred to as the “fourth artificial neural network”) together used in step 130. Each of these networks may be implemented as 7-layer Equivariant Graph Neural Networks (EGNNs) with a hidden dimension of 128 as described in Satorras et al. (Victor Garcia Satorras, Emiel Hoogeboom, and Max Welling. E(n) equivariant graph neural networks. arXiv preprint arXiv:2102.09844). The EGNNs produce embeddings for each point in the input graph, which can be aggregated into a global graph representation using sum-pooling.

The model (e.g., the group of neural networks) can be trained to autoregressively predict the next atom types (i.e., the different chemical elements appearing in the dataset), next edge types (i.e., bond type, or explicit indication of a lack of a bond), next atom distances, and next atom angles for sequentially presented subgraphs of training molecules.

Without being bound to the following motivation and/or derivation, it is instructive to consider a probabilistic model for the 3D graphs of molecules described above. As introduced above, a molecule can be represented as a 3-dimensional graph G=(V, A, X). For a molecule with n atoms, V∈ [symbol] is a list of d-dimensional atom features, A∈ [symbol] is an adjacency matrix with b-dimensional edge features, and X∈ [symbol] is a list of 3D atomic coordinates for each atom. In practice, V encodes the atomic number of each atom, and A encodes the number of shared electrons in each covalent bond. To model a chemical space of interest, a goal is to learn a probability distribution p(V, A, X) over the chemical space. One approach to learning this distribution is to form various marginal and conditional densities with respect to this joint distribution. For example, a graph-based generative models can learn the marginal distribution

$$p ( V , A ) = {\int }_{X} p ( V , A , X ) dX ,$$

molecular geometry prediction amounts to learning the conditional distribution p(X|V, A), and 3D generative models (e.g., G-SchNet) learn the distribution

$$p ( V , X ) = {\int }_{A} p ( V , A , X ) dA .$$

To learn the joint distribution p(V, A, X), it can be effective to factorize the density. For instance, the following factorization can be used:

$$p ( V , A , X ) = {{\prod }_{i = 1}}^{n} p ( {V}_{: i} , {X}_{: i} , {A}_{: j} \vert {V}_{: i - 1} , {A}_{: i - 1} , {X}_{: i - 1} ) \cdot p ( stop \vert V , A , X ) = {{\prod }_{i = 1}}^{n} p ( {X}_{: i} \vert {V}_{: i} , {A}_{: i} , {X}_{: i - 1} ) p ( {A}_{: i} \vert {V}_{: i} , {A}_{: i - 1} , {X}_{: i - 1} ) p ( {V}_{: i} \vert {V}_{: i - 1} , {A}_{: i - 1} , {X}_{: i - 1} ) \cdot p ( stop \vert V , A , X )$$

Here, n is the number of atoms in the input graph, and V_{:i}, A_{:i} and X_{:i} indicate the graph (V, A, X) restricted to the first i atoms. Computing p (V_{:i}|V_{:i-1}, A_{:i-1}, X_{:i-1}) amounts to predicting a single atom type based on a 3D graph (V_{:i-1}, A_{:i-1}, X_{:i-1}). Calculating p(A_{:i}|V_{:i}, A_{:i-1}, X_{:i-1}) is more complex because it involves a prediction over a new row of the adjacency matrix. More concretely, computing the conditional density of A_{:i}∈R^{i×i×b} amounts to computing a joint density over the new entries of the adjacency matrix A_{i,1}, . . . , A_{i,i-1}∈R^{b}. To solve this problem, this distribution is further decomposed as:

$$p ( {A}_{: i} \vert {V}_{: i} , {A}_{: i - 1} , {X}_{: i - 1} ) = p ( {A}_{i , 1} , … , {A}_{i , i - 1} \vert {V}_{: i} , {A}_{: i - 1} , {X}_{: i - 1} ) = {{\prod }^{i - 1}}_{j = 1} p ( {A}_{i , j} \vert {A}_{i , : j - 1} , {V}_{: i} , {A}_{: i - 1} , {X}_{: i - 1} )$$

Intuitively, A_{i,1}, . . . , A_{i,i-1} represent the edges from atom i to atoms 1 . . . i−1.

Finally, estimating the density p(X_{:i}|V_{:i}, A_{:i}, X_{:i-1}) involves modeling a continuous distribution over positions Xᵢ∈ [symbol] for atom i. To accomplish this, if Xᵢ is assumed to belong to a finite set of points χ, and its probability distribution is modeled as a product of distributions over angles and interatomic distances:

$$p ( {X}_{i} \vert {V}_{: i} , {A}_{: i} , {X}_{: i - 1} ) = \frac{1}{C} {{\prod }_{j = 1}}^{i - 1} p \text{} ( ‖ {X}_{i} - {X}_{j} ‖ \vert {V}_{: i} , {A}_{: i} , {X}_{: i - 1} ) \cdot {\prod }_{( j , k ) ∈ I} p ( Angle ( {X}_{i} - {X}_{k} , {X}_{j} - {X}_{k} ) \vert {V}_{: i} , {A}_{: i} {X}_{: i - 1} )$$

Intuitively, p(∥Xᵢ−Xⱼ∥|V_{:i}, A_{:i}, X_{:i-1}) predicts the distances from each existing atom to the new atom, and p (Angle (Xᵢ−Xₖ, Xⱼ−Xₖ)|V_{:i}, A_{:i}, X_{:i-1}) predicts the bond angles of connected triplets of atoms involving atom i. I is a set of pairs of atoms where atom k is connected to atom i, and atom j is connected to atom k. “Angle” denotes the angle between two vectors. C is a normalizing constant derived from summing this density over all of χ. To increase the computational tractability of estimating this factorized density, we assume that the nodes in the molecular graph (V, A, X) are listed in the order of a breadth-first traversal over the molecular graph.

In order to predict the geometry of a specific molecular graph, Dijkstra's algorithm can be used to search for geometries of those molecules that are assigned a high likelihood. In such an approach, the given molecular graph is unrolled in a breadth-first order, so predicting the molecule's geometry amounts to determining a sequence of positions for each atom during the rollout. If atomic positions are discretized, then the space of possible molecular geometries forms a tree. Each edge in the tree can be assigned a likelihood by the system. Predicting a plausible geometry thus amounts to finding a path where the sum of the log-likelihoods of the edges is large. This can be accomplished using a graph search algorithm such as A* or Dijkstra's algorithm. The geometry prediction algorithm is presented in Algorithm 1 in the Appendix. This procedure has been found to be effective and computational feasible for molecules in GEOM-QM9 (described further below).

A preferred implementation uses a collection of the four equivariant neural networks described above implemented in software instructions for execution on a general purpose processor (e.g., “CPU”) or special purpose or parallel processor (e.g., a graphics processing unit, “GPU”) or optionally using at least some special-purpose circuitry. The neural networks are configurable with quantities (often referred to as “weights”) that are used in arithmetic computations within the neural networks.

As introduced above, each of these networks is implemented as a 7-layer EGNN with a hidden dimension of 128. An EGNN network takes in a 3D graph as input, and outputs vector embedding for each node in the input graph. The system also uses four relatively simple Multi-Layer Perceptrons (MLPs) D_{A}, D_{E}, D_{D}, and D_{θ} to decode the output embeddings of each EGNN into softmax probabilities. The following subnetworks are used to compute the components of the factorized density above as follows:

$$p ( {V}_{: i} \vert {V}_{: i - 1} , {A}_{: i - 1} , {X}_{: i - 1} ) = Softmax ( {D}_{A} ( SumPool ( {F}_{A} ( {V}_{: i - 1} , {A}_{: i - 1} , {X}_{: i - 1} ) ) ) )$$

$$p ( {A}_{i , j} \vert {A}_{i , : j - 1} , {V}_{: i} , {A}_{: i - 1} , {X}_{: i - 1} ) = Softmax ( {D}_{E} ( {{F}_{E} ( {A}_{i , : j - 1} , {V}_{: i} , {A}_{: i - 1} , {X}_{: i - 1} )}_{j} ) )$$

$$p ( ‖ {X}_{i} - {X}_{j} ‖ \vert {V}_{: i} , {A}_{: i} , {X}_{: i - 1} ) = Softmax ( {D}_{D} ( {{F}_{D} ( {V}_{: i} , {A}_{: i} , {X}_{: i - 1} )}_{j} ) )$$

$$h = {F}_{θ} ( {V}_{; i} , {A}_{: i} , {X}_{: i - 1} )$$

$$p ( Angle ( {X}_{i} - {X}_{k} , {X}_{j} - {X}_{k} ) \vert {V}_{: i} , {A}_{: i} , {X}_{: i - 1} ) = Softmax ( {D}_{θ} ( {h}_{j} , {h}_{k} ) )$$

Note that the predicted distance and angle distributions are discrete softmax probabilities. These discrete distributions correspond to predictions over equal-width distance and angle bins. Because all of the EGNN-computed densities are insensitive to translations and rotations of the input graph, the full product density is also insensitive to these transformations.

At training time, for each training molecule, a breadth-first decomposition of a graph (V, A, X) is computed. The subnetworks are trained to autoregressively predict the next atom types, edges, distances, and angles in this decomposition according to the model described above. A cross entropy losses is used to penalize the model for making predictions that deviate from the actual next tokens in the breadth-first decomposition. While the model's density is not invariant across different breath-first decompositions of the same molecule, resampling each molecule's decomposition at every epoch enables the model to learn to ascribe equal densities to different rollouts of the same molecule.

The training algorithm is also provided in detail in the Appendix. Experimental evaluation used the Adam optimizer with a base learning rate of 0.001. All models were able to train in approximately one day on a single NVIDIA A100 GPU. The model is trained using teacher forcing, so it only learns to make accurate predictions when given well-formed structures as autoregressive inputs. To increase geometric robustness a uniform random noise of up to 0.05 Å is added to the atomic coordinates during training for all datasets.

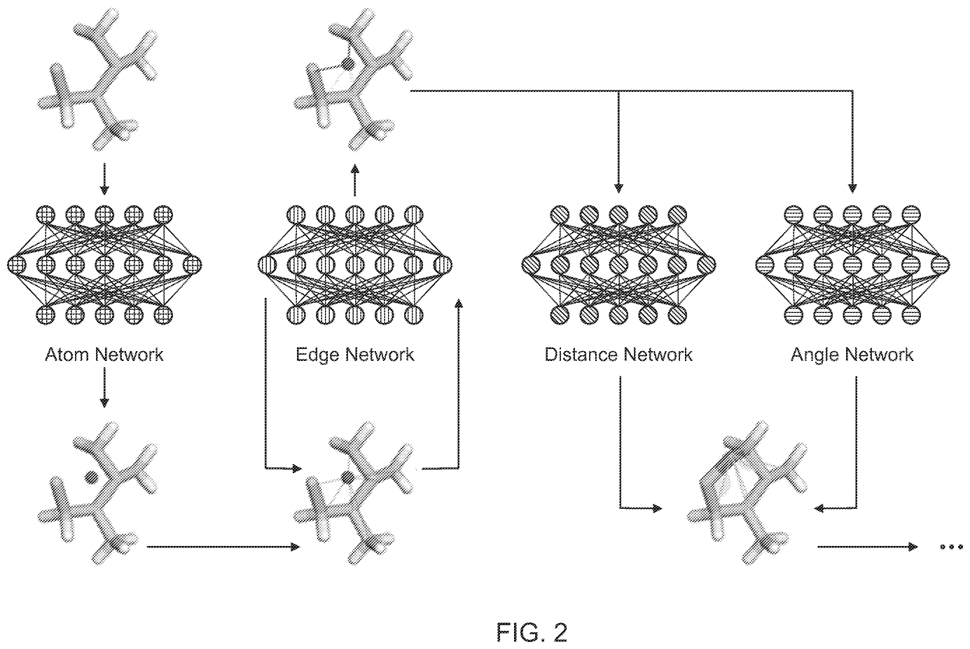

To sample a 3D molecule from a trained model, a single initial atom or a larger molecular fragment is started with. First, the atom network computes a discrete distribution over new atom types to add, from which a new atom type can be sampled multinomially. The edge network is then used to sequentially sample the edge types joining the new atom to each of the previously generated atoms. The distance and angle networks compute distributions over interatomic distances and bond angles involving the newly sampled atom. To sample the new atom's position, we construct the discrete set of points χ as a fine grid surrounding the previously generated atoms, and assign each point a probability according to the model's distance and angle predictions. Finally, the new atom's position is sampled multinomially from the set χ. The resulting molecular graph, which has been extended by one atom, is then fed back into the autoregressive sampling procedure until a stop token is generated. This sampling process by which an atom is randomly added (i.e., by process 100 of FIG. 1) to a partial molecule is illustrated in FIG. 2.

As discussed above, the system makes use of an autoregressive model that incrementally adds to a partially completed molecular graph. We denote a partially completed graph with n atoms as Gₙ=(Vₙ, Aₙ, Xₙ). V∈R^{n×d} is a list of one-hot encoded atom types (i.e., the different chemical elements appearing in the dataset), and d is the number of possible atom types. Aₙ∈R^{n×n×b} is an adjacency matrix recording the one-hot encoded bond type between each pair of atoms, with b representing the number of bond types. Xₙ∈R^{n×3} is a list of atom positions. For the adjacency matrix Aₙ, an extra bond type is included indicating that the atoms are not chemically bonded (unbonded atoms are still connected in the sense that information can propagate between them during the EGNN computation).

The addition of a new atom proceeds in three steps. First, a new atom type is selected as follows:

$${H}_{n} = SumPool ( {F}_{A} ( {G}_{n} ) )$$

$${a}_{n + 1} ∼ Categorical ( Softmax ( {D}_{A} ( {H}_{n} ) ) ) ,$$

where aₙ₊₁ is the type of the new atom, and D_{A} is a neural network that decodes the EGNN graph embedding into a set of softmax probabilities. The network D_{A} is implemented as a 3-layer MLP. Note that, in addition to all of the atom species in the training set, we allow aₙ₊₁ to take on an extra “stop token” value. If this value is generated, the molecule is complete, and generation terminates.

The next step in the generation procedure is to connect the new atom to the existing graph with edges. We do this in a similar manner to GraphAF, and query every atom sequentially to determine its new bond type, updating the adjacency list as needed. More formally, this procedure works as follows:
Initialize Eₙ∈R^{n×b} as a matrix containing each atom's edge type to the new atom aₙ₊₁. At initialization, let Eₙ contain all unbonded edge types.
for i in 1 . . . n do:
   V′ₙ=Concat (Vₙ, Eₙ, OneHot(aₙ₊₁)). V′ₙ is a n×(2d+b) matrix of modified atom features. Row i contains the one-hot encoded type of atom i, the one-hot encoded type of atom i's current edge to atom n+1, and the one-hot encoded type of atom n+1.

$${G}_{n}^{′} = ( {V}_{n}^{′} , {A}_{n} , {X}_{n} )$$

$${h}_{i} = {F}_{E} {( {G}_{n}^{′} )}_{i}$$

bᵢ˜Categorical Softmax (D_{E} (hᵢ))). bᵢ is a sampled bond type between atom i and atom n+1. D_{E} is another MLP decoder which acts on the node-specific embedding of atom i.
Eₙ[i:]←OneHot (bᵢ)

Through this procedure, a set of bonds is sampled for the new atom.

In the final step, the new atom is given a 3D position. This is accomplished by predicting a discrete distribution of distances from each atom in the graph to the new atom, and a discrete distribution of bond angles between edges that contain the new atom and all adjacent edges. These predictions induce a distribution over 3D coordinates. In a secondary step, we approximately sample from this spatial distribution by drawing points from a fine, stochastic 3D grid using the likelihood function given by the distance and angle predictions. More formally, the positions of the atoms are predicted as follows:

$${V}_{n}^{′} = Concat ( {V}_{n} , {E}_{n} , OneHot ( {a}_{n + 1} ) )$$

$${G}_{n}^{′} = ( {V}_{n}^{′} , {A}_{n} , {X}_{n} )$$

$${h}_{1} , … , {h}_{n} = {F}_{D} ( {G}_{n}^{′} )$$

$${h}_{1}^{′} , … , {h}_{n}^{′} = {F}_{0} ( {G}_{n}^{′} )$$

$${p}_{i} = Softmax ( {D}_{D} ( {h}_{i} ) ) for i = 1 .. n$$

$${q}_{i j} = Softmax ( {D}_{θ} ( {h}_{i}^{′} , {h}_{j}^{′} ) )$$

$$for i , j s . t . argmax ( {A}_{n} [ i , j ] ) > 0 and argmax ( {E}_{n} [ j ] ) > 0 ,$$

where D_{D} and D_{θ} are MLP decoders as before. Note that the matrix Eₙ is re-used from the edge prediction step, which has accumulated all of the new edges to atom n+1. The probability vectors p₁, . . . , pₙ now define discrete distributions over the distances between each atom in the graph and the new atom, and the vectors qᵢⱼ define distributions over bond angles. These distributions can be treated as being independent, so that the product rule can be used to compute the likelihood of any point in 3D space:

$${L}_{D} ( {x}_{n + 1} ) = {\prod }_{i = 1}^{n} {p}_{i} ( ‖ {x}_{n + 1} - {x}_{i} ‖ )$$

$${L}_{θ} ( {x}_{n + 1} ) = {\prod }_{i , j ∈ I} {q}_{i j} ( Angle ( {x}_{n + 1} - {x}_{j} , {x}_{i} - {x}_{j} ) )$$

$$L ( {x}_{n + 1} ) = {L}_{D} ( {x}_{n + 1} ) \times {L}_{θ} ( {x}_{n + 1} ) ,$$

where xᵢ is the location of atom i, I is the set of incident edges to the neighbors of aₙ₊₁, and “Angle” denotes the angle between two vectors. To sample a point from the likelihood L(xₙ₊₁), we simply assign a likelihood to every point in a fine, stochastic grid surrounding the atoms that are bonded to aₙ₊₁, and sample from it as a categorical distribution to produce a new spatial location.

By repeating this procedure until termination, the system can produce a 3D molecule from a single starting atom. Note that, because the generation process is sequential, it is possible to mask out atom or edge selections that would violate valence constraints, thereby guaranteeing that generated molecules follow basic chemical rules. It is also possible for the model to predict a non-terminating atom, but then predict that no edges connect to that atom. The edge sampling procedure is re-run until at least one edge to the new atom is generated. In one practice, if no edge to the new atom is produced after 10 resampling attempts, the new atom is discarded and the generation process is said to have terminated.

The approaches described above were evaluated by training the system to generate 3D molecules from three datasets: QM9, GEOM-QM9 and GEOM-Drugs (Raghunathan Ramakrishnan, Pavlo O. Dral, Matthias Rupp, von Lilienfeld, and O. Anatole. Quantum chemistry structures and properties of 134 kilo molecules. Scientific Data, 1(140022), 2014; and Simon Axelrod and Rafael Gómez-Bombarelli. Geom: Energy-annotated molecular conformations for property prediction and molecular generation. arXiv preprint arXiv:2006.05531, 2020). QM9 contains 134,000 small molecules with up to nine heavy atoms (i.e., not including hydrogen) of the chemical elements C, N, O, and F. Each molecule has a single set of 3D coordinates obtained via Density Functional Theory calculations, which approximately compute the quantum mechanical energy of a set of 3D atoms in space. GEOM-QM9 contains the same set of compounds as QM9, but with multiple geometries for each molecule. GEOM-Drugs also has multiple geometries for each molecule, and contains over 300,000 drug-like compounds with more heavy atoms and atomic species than QM9.

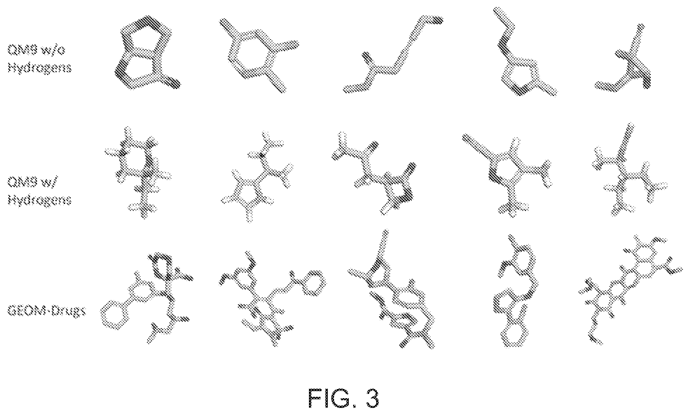

On QM9, one version of the model was trained with heavy atoms only, and one version with hydrogens. To ensure the quality of the geometric data, OpenBabel (O'Boyle et al., 2011) was used to convert the coordinates from the QM9 source files into SDF files, which contain both coordinates and connectivity information inferred based on inter-atomic distances. All molecules for which the inferred connectivity did not match the intended SMILES string from the QM9 source data were discarded, leaving approximately 124,000 molecules with SDF-formatted bonding information. Approximately 100,000 of these molecules were used for training, with the additional 24,000 molecules held out for validation. GEOM-QM9 was trained on 200,000 molecule-geometry pairs, and excluded all SMILES strings from the test set of Xu et al. (2021b). For GEOM-Drugs training only used heavy atoms, using 50,000 randomly chosen molecule-geometry pairs for training. It was found that, after 60 epochs of training, the system was able to generate highly realistic 3D molecules from all of these datasets. Visualization samples from QM9 and GEOM-Drugs are shown in FIG. 3.

An assessment of the quality of generated molecules included analyzing the characteristics of generated molecular graphs on QM9. In particular, the percentages of novel and unique molecular graphs generated by the heavy atom QM9 model in a sample of 10,000 molecules were assessed. A novel molecular graph is defined as a graph not present in the training data. The uniqueness rate is defined by the number of distinct molecular graphs generated, divided by the total number of molecules generated. Using the results for novelty, validity, and uniqueness metrics, the present approach is compared against GraphAF and CGVAE, which are two recently published molecular graph generators that also add one atom at a time. The results also compared against a geometry-unaware baseline that was created by removing the geometric networks from the system and setting all positional inputs to 0. These results are reported in the following table:

| | | Validity | | | Passed |
|---|---|---|---|---|---|
| Model | Validity | (w/o check) | Uniqueness | Novelty | Strain Check |
| CGVAE | 100% | 49.55% | 97.09% | 88.18% | 70.84% |
| GraphAF | 100% | 67% | 94.51% | 88.93% | — |
| GEN3D- | 100% | 99.79% | 95.59% | 25.93% | 90.97% |
| GEN3D | 100% | 98.80% | 94.33% | 33.18% | 93.36% |
| QM9 | 100% | 100% | — | — | 92.41% |
| (truth) | | | | | |

Even without imposing checks at generation time, the system produced molecules that obey valence constraints 98.8% of the time after training on QM9. This far exceeds the unchecked validity rate of 67% achieved by GraphAF, suggesting that the present approach has a better understanding of basic rules of chemistry. Interestingly, the geometry-free baseline achieves 99.8% validity, suggesting that improvements in chemical validity come from architectural differences that may be unrelated to the generation of 3D geometries. The system achieves a uniqueness rate of 94.3%, which is similar to the rates for GraphAF and CGVAE. The geometric feasibility of generated graphs was assessed by converting them into 3D coordinates using CORINA (Sadowski & Gasteiger, 1993), and then computing the volume of the tetrahedron enclosed by each sp3 tetrahedral center, with vertices located 1 Å along each tetrahedral bond. Graphs that could not be converted with CORINA, or contained tetrahedral centers with volumes less than 0.345 Å³, were classified as being overly strained. The system produced fewer overly strained molecules than other models, including the geometry-free baseline, suggesting that explicitly generating molecular geometries helps bias the model towards stable compounds.

Further assessment addressed the quality of the 3D geometries produced by the present GEN3D system. The generated molecules were compared to ENF and G-SchNet, which are the only other published models that generate samples from the distribution of 3D QM9 molecules. Both ENF and G-SchNet produce the positions of heavy atoms and hydrogens as the output of their generative process. In order to facilitate a direct comparison, these models were compared to the present all-atom QM9 model. The ENF paper reports atomic stability as the percentage of atoms that have a correct number of bonds, and molecular stability as the fraction of all molecules with the correct number of bonds for every atom. These metrics are shown in the table below, which compares the present GEN3D system to ENF, G-SchNet, and related baselines.

| | Atom | Mol | Distance |
|---|---|---|---|
| Model | Stability | Stability | JS |
| G-SchNet | 98.7% | 77.0% | .0031 |
| GEN3D Focus Atom | 98.7% | 82.0% | .0037 |
| GEN3D No Angles(w/o check, OpenBabel | 99.4% | 93.4% | .0030 |
| edges) | | | |
| GEN3D No Angles (w/o check, GEN3D | 99.6% | 95.7% | .0030 |
| edges) | | | |
| GEN3D No Angles (w/check, OpenBabel | 99.5% | 95.5% | .0030 |
| edges) | | | |
| GEN3D No Angles (w/check, GEN3D | 99.7% | 98.0% | .0030 |
| edges) | | | |
| GEN3D (w/o check, OpenBabel edges) | 99.6% | 96.4% | .0014 |
| GEN3D (w/o check, GEN3D edges) | 99.7% | 97.5% | .0014 |
| GEN3D (w/check, OpenBabel edges) | 99.75% | 97.6% | .0014 |
| GEN3D (w/check, GEN3D edges) | 99.87% | 99.1% | .0014 |
| QM9 (truth) | 99.99% | 99.9% | 0 |

GEN3D outperformed all other models, achieving 97.5% molecular stability without any valence masking, compared to 77% for G-SchNet and 4.3% for ENF. In order to assess the geometric realism of the generated molecules, the authors of ENF computed the Jensen-Shannon divergence between a normalized histogram of inter-atomic distances and the true distribution of pairwise distances from the QM9 dataset. This metric was also computed and it was found that GEN3D advances the state of the art, reducing the JS divergence by a factor of two over G-SchNet and a factor of four over ENF. The fact that GEN3D substantially outperforms ENF and G-SchNet, both of which only generate coordinates and do no generate bonding information, suggests that generating bonds as well as coordinates significantly increases the quality of generated molecules.

To confirm this, a systematic ablation study was conducted in which the angle and edge networks of GEN3D were successively removed to produce the baseline model that is very similar to G-SchNet. It was found that performance in both geometric and chemical accuracy metrics dropped continuously as these features were removed, and that the baseline model performed very similarly to G-SchNet. In addition, GEN3D is much less computationally expensive to train than ENF's flow-based generative process, and it is applicable to larger drug-like molecules. These comparisons are reported in the table below, and the true and learned histograms of pairwise distances are plotted in FIG. 5. In order to be consistent with the ENF paper, the Jensen-Shannon divergence was only computed between generated and QM9 molecules with exactly 19 total atoms.

| Model | Atom Stability | Mol Stability | Distance JS |
|---|---|---|---|
| GNF-attention | 72.0% | 0.3% | .007 |
| E-NF | 84.0% | 4.2% | .006 |
| G-SchNet | — | 77.0% | — |
| GEN3D (w/o check) | 99.7% | 97.5% | .0014 |
| GEN3D (w/check) | 99.87% | 99.1% | .0014 |
| QM9 (truth) | 99.99% | 99.9% | 0 |

The Jensen-Shannon divergence metric provides confidence that GEN3D is, on average, generating accurate molecular geometries. This metric, however, is relatively insensitive to the correctness of individual molecular geometries because it only compares the aggregate distributions of distances. In order to further validate the accuracy of GEN3D's generated geometries, the system models were used to predict the geometries of specific molecular graphs, and compared its accuracy with purpose-built tools designed for molecular geometry prediction, such as the model described in Xu et al. (2021b). This evaluation amounts to verifying the accuracy of the conditional distribution p(X|V, A) when the joint distribution p(V, A, X) is learned by GEN3D. We approximated this conditional distribution by using a search algorithm to identify geometries X that give a high value to p(V, A, X) as calculated by GEN3D when V and A are known inputs.

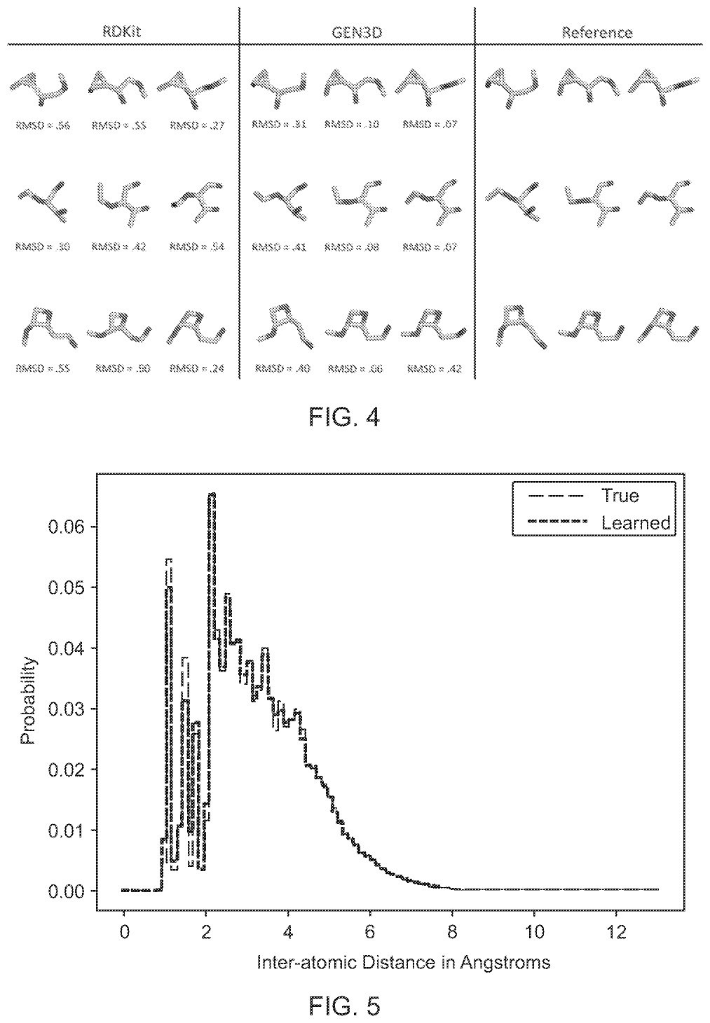

To evaluate the ability of GEN3D to predict molecular geometries, GEN3D was trained to generate molecules from GEOM-QM9 (Axelrod & Gómez-Bombarelli, 2021). We then followed the evaluation protocol described in Xu et al. (2021a) and Xu et al. (2021b) with the same set of 150 molecular graphs, which were excluded from the training set. As in these prior works, an ensemble of geometries were predicted and then computed COV and MAT scores with respect to the test set. The COV score measures what fraction of reference geometries have a “close” neighbor in the set of generated geometries, where closeness is measured with an aligned RMSD threshold. A threshold of 0.5 Å was used, following Xu et al. (2021b). The MAT score summarizes the aligned RMSD of each reference geometry to its closest neighbor in the set of generated geometries (for additional detail on the evaluation protocol, see Xu et al. (2021a)). GEN3D achieves results that are among the best for published models on both metrics. In particular, its MAT scores outperform all prior methods that do not refine geometries using a rules-based force field. GEN3D was compared with previous machine learning models for molecular geometry prediction, as well as the ETKDG algorithm implemented in RDKit (which predicts molecular geometries using a database of preferred torsional angles and bond lengths (Riniker & Landrum, 2015)). The following table shows the results of this evaluation, and FIG. 4 visualizes representative geometry predictions. The results in the table indicate that GEN3D is accurately sampling from the joint distribution of molecular graphs and molecular geometries.

| | |
|---|---|
| COV (%) | MAT (Å) |

| Metric | Mean | Median | Mean | Median |
|---|---|---|---|---|
| GraphDG | 55.09% | 56.47% | 0.4649 | 0.4298 |
| CGCF | 69.60% | 70.64% | 0.3915 | 0.3986 |
| ConfVAE | 77.98% | 82.82% | 0.3778 | 0.3770 |
| RDKit | 80.68% | 87.50% | 0.3349 | 0.3245 |
| GEN3D | 73.62% | 77.14% | 0.3168 | 0.3049 |

The approaches described above were also evaluated for their ability to generate 3D molecules in poses that have favorable predicted interactions with a target protein pocket, as evaluated by the Rapid Overlay of Chemical Structures (ROCS) in virtual screening algorithm (see, e.g., J Andrew Grant, et al. A fast method of molecular shape comparison: A simple application of a gaussian description of molecular shape. Journal of Computational Chemistry, 17(14):1653-1666, 1996). In this evaluation, a model was trained on GEOM-drugs (this model is denoted GEN3D-gd). A large pre-existing library of 62.9-million compounds was curated, containing up to 250 molecular geometries for each compound generated with OpenEye Omega (Emanuele Perola and Paul S Charifson. Conformational analysis of drug-like molecules bound to proteins: an extensive study of ligand reorganization upon binding. Journal of Medicinal Chemistry, 47(10):2499-2510, 2004), and screened the resulting 13.8-billion conformations against our target pocket using ROCS. The top 1000 scoring geometries belonging to distinct molecular graphs were selected from the library, and GEN3D-gd was fine-tuned on these 1000 molecules for 100 epochs (this model is denoted GEN3D-ft).

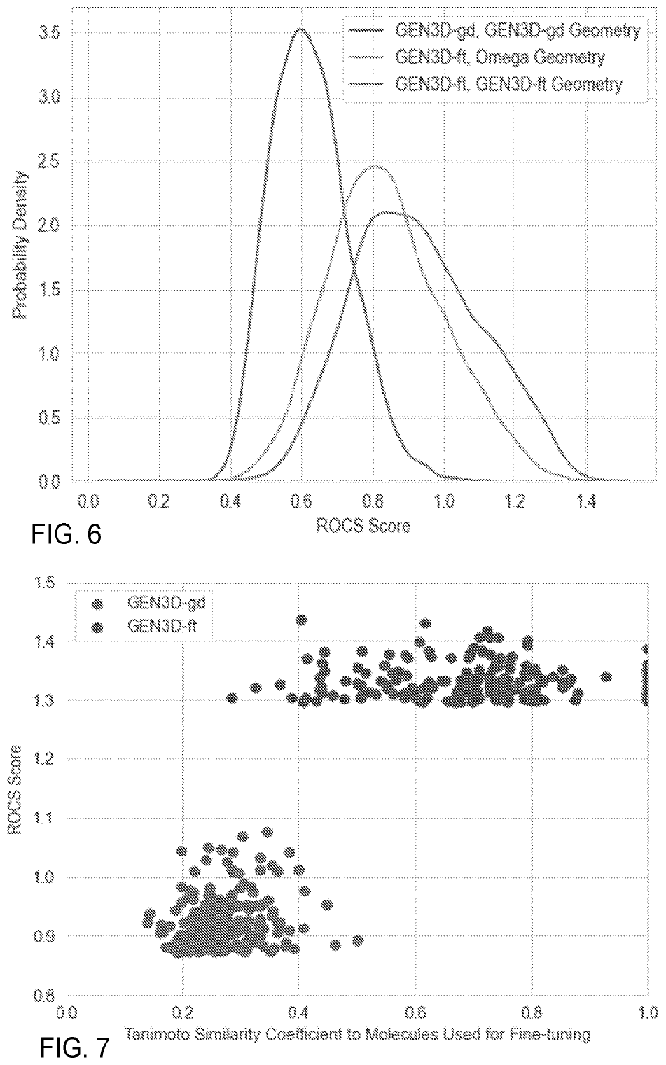

To evaluate the ability of the system to learn chemical and geometric features that are conducive to binding the pocket, 10,000 molecules were generated with 3D coordinates from GEN3D-gd and GEN3D-ft. The molecular graphs were generated by GEN3D-ft and recalculated molecular geometries for them using OpenEye Omega. The molecules generated by GEN3D-ft were excluded if the molecular graph overlapped with the fine-tuning set (2.07% of the total), and scored the remainder using ROCS. The fine-tuning significantly increased the scores of generated compounds. Because GEN3D-ft was fine-tuned on high-scoring molecular geometries, the molecular geometries it generated implicitly include information about the target geometry that were unavailable to GEN3D-gd and OpenEye Omega. As a result, the scores for GEN3D-ft geometries were, on average, better than those generated by other methods. These results are shown in FIG. 6.

Ideally, this training procedure would allow the models to generate strong binders that are significantly different from those in the fine-tuning set. To compare each model's ability to produce both high-quality and novel compounds, the top 2% of molecules generated by each model were picked by ROCS score and plotted their ROCS scores against their maximum Tanimoto similarity coefficient (also called a Jaccard coefficient of community) to an element of the set used for fine-tuning. The Tanimoto similarity coefficient ranges from fully dissimilar at 0.0 to identical at 1.0, and is a measure of the structural closeness of two molecular graphs. It is computed by representing two molecules with Extended-Connectivity Fingerprints, which are essentially lists of activated bits corresponding to substructures present in each molecule. Here, RDKit's implementation of Morgan fingerprints were used with 2048 bit radius 2, and without chirality.

The results show that GEN3D-ft generated molecules with high ROCS scores across a wide range of Tanimoto similarities to the fine-tuning set. In this particular instance, the highest ROCS scoring molecule generated by GEN3D-ft had a Tanimoto similarity to the fine-tuning set of about 0.4. Molecules generated by GEN3D-ft had significantly higher scores than those generated by GEN3D-gd, even when comparing molecules from each model with comparable similarities to the fine-tuning set. These results are shown in FIG. 7.

These experiments indicate that GEN3D is able to shift its generative distribution into specific regions of chemical and geometric space.

It should be understood that a number of alternatives are within the scope of the following claims. For example, the particular decomposition used and/or the particular forms of machine-learning models may be changed. While maintaining an autoregressive process of incremental addition to a particular molecule, a next increment may be bonded to atoms in a partial molecule and placed with respect to that partial molecule using an integrated approach, such as a combined neural network model. Furthermore, as introduced above, the approach covers addition of groups of multiple atoms in one increment, and these groups may be discovered, or may be precomputed as representing a “library” of increments that can be used in addition or instead of simpler one-atom increments. In some examples, as increments are added, the geometric configuration of the entire partial molecule may be recomputed rather than simply determining geometric information for the newly added increment. In some examples, rather than only adding to a molecule, other “edits” to a partial molecule may be used, for example, removal of previously-added atoms, while maintain the incremental construction of an overall molecule.

As previously introduced the approaches described above may be implemented using software instructions, which may be stored on non-transitory machine-readable media, for execution on a general purpose processor (e.g., “CPU”) or special purpose or parallel processor (e.g., a graphics processing unit, “GPU”). Optional at least some special-purpose circuitry may be used, for example, for runtime (molecule generation) or training (model configuration) stages. It is not necessary that the runtime processing necessarily use the same processors or hardware infrastructure as the training, and training may be performed in multiple steps, each of which may also be performed on different processors and hardware infrastructure.

A number of embodiments of the invention have been described. Nevertheless, it is to be understood that the foregoing description is intended to illustrate and not to limit the scope of the invention, which is defined by the scope of the following claims. Accordingly, other embodiments are also within the scope of the following claims. For example, various modifications may be made without departing from the scope of the invention. Additionally, some of the steps described above may be order independent, and thus can be performed in an order different from that described.

```
APPENDIX
Algorithm 1 Molecular Geometry Search using GEN3D
Input: A molecular graph G = (V, A) and a number of iterations T.
Perform a Breadth-First Search on G, producing a series of vertex lists V₁, ..., V_{N} and adjacency
matrices A₁, ..., A_{N}.
Q ← Empty PriorityQueue( )
Q.push((0. 1, [0.0, 0.0, 0.0])) [symbol]   Queue entries have the form (Negative Log Likelihood,
                                              Number of Atoms, Position Matrix)
t ← 0
results ← [ ]
while t < T do
  nll, i, X ← Q.dequeue( )
  if i = = N then
    results.append((nll, X))
  else
    for j in {1..i} do [symbol]   Find an atom which is a neighbor of the next atom in the rollout
      if argmax(Aᵢ₊₁[i + 1, j]) > 0 then
        neigh ← j
      end if
    end for
    points ← MakeGrid(X [neigh]) [symbol]  Create a fine grid of potential positions around the
                                  neighbor atom
    for x in points do
      dst_lik = p_{D}(x|Vᵢ₊₁, Aᵢ₊₁, Xᵢ)
      ang_lik = p [symbol] (x|Vᵢ₊₁, Aᵢ₊₁, Xᵢ)
      atm_lik = p_{A}(Vᵢ₊₂|Vᵢ₊₁, Aᵢ₊₁, [Xᵢ; x])
      edg_lik = p_{E}(Aᵢ₊₂|Vᵢ₊₂, Aᵢ₊₁, [Xᵢ; x])
      nll ← nll - log(dst_lik) - log(ang_lik) - log(atm_lik) - log(edg_lik)
      Q.push((nll, i + 1, X; x]))
    end for
  end if
end while
return results
Algorithm 2 Training GEN3D
Initial: Parameters ϕ of GEN3D subnetworks F_{A}, D_{A}, F_{E}, D_{E}, F_{D}, D_{D}, F [symbol] , D [symbol] .
while ϕ not converged do
  Sample a 3D molecule G = (V, A, X ) from the training set.
  Perform a Breadth-First search on G, producing a series of vertex lists V₁, ..., Vₙ.
  adjacency matrices A₁, ..., Aₙ, and 3D coordinates X₁, ..., Xₙ.
  Let Gᵢ = (V_{i,} Xᵢ, Aᵢ) denote the partially complete graph at step i of the BFS.
  Let a, and xᵢ for i ϵ {1..N} be the type and position of the atom visited in iteration i of the
  BFS. In addition, let [symbol] _{N+1} be a special “stop token” atom type.
  Finally, let Eᵢ ϵ E [symbol]   be a matrix with the one-hot encoded edge types connecting each of
  atomas 1..i - 1 to atom i.
  for i in (1..N} do
    atom_probs ← Softmax(D_{A}(SumPool(F_{A}(Gᵢ))))
    loss ← CrossEntropy (atom_probs, aᵢ+1)
    if i < N then
      edge_probs ← Zeros(i, b)
      edge_accum ← Zeros(i, b)
      edge_accum[i, 0] ← 1
      for j in {1..i} do
        edge_accum[ [symbol]  j] ← Eᵢ[ [symbol]  j]
        G [symbol]  ← (Concat(Vᵢ, OneHot(aᵢ₊₁), edge_accum), Aᵢ, Xᵢ)
        h_edge ← D_{E}(F_{E}(G [symbol] ))
        edge_probs[j] ← Softmax(h_edge)[j])
      end for
      loss ← loss + CrossEntropy(edge_probs, Eᵢ)
      G [symbol]  ← (Concat(Vᵢ, OneHot(aᵢ₊₁), Eᵢ), Aᵢ, Xᵢ)
      dists ← ||xᵢ₊₁ - Xᵢ|| [symbol]   dists ϵ [symbol]  . Distances to the new atom.
      dist_probs ← Sofmax(D_{D}(F_{D}(G [symbol] ))) [symbol]   dist_probs ϵ [symbol]  . Predicted bins.
      loss ← loss + CrossEntropy(dist_probs, make_bins(dists))
      h_angle ← F [symbol] +(G [symbol] )
      angle_loss ← 0
      angle_count ← 0
      for j in {1..i} do
        for k in {1..j) - 1} do
          if argmax(Aᵢ[j, k]) > 0 and argmax(Eᵢ[j]) > 0 then
            ange_probs ← Softmax (D [symbol] (Concat(h_angle[j], h_angle[k])))
            angle ← Angle(Xᵢ[k] - Xᵢ[j], xᵢ₊₁ - Xᵢ[j])
            angle_loss ← angle_loss + CrossEntropy(angle_probs, make_bins(angle))
            angle_count ← angle_count + 1
          end if
        end for
      end for
      loss ← loss + angle_loss/angle_count
    end if
    ϕ ← ϕ - η∇ [symbol] loss
  end for
end while [symbol]
indicates data missing or illegible when filed
```

### REFERENCE CITED IN THE DESCRIPTION

Simon Axelrod and Rafael Gómez-Bombarelli. Geom: Energy-annotated molecular conformations for property prediction and molecular generation. arXiv preprint arXiv:2006.05531, 2020.
Noel O'Boyle, Michael Banck, Craig James, Chris Morely, Tim Vandermeersch, and Geoffrey Hutchison. Open Babel: An open chemical toolbox. Journal of Cheminformatics, 3(33), 2011.
Raghunathan Ramakrishnan, Pavlo O. Dral, Matthias Rupp, von Lilienfeld, and O. Anatole. Quantum chemistry structures and properties of 134 kilo molecules. Scientific Data, 1(140022), 2014.
Jens Sadowski and Johann Gasteiger. From atoms and bonds to three-dimensional atomic coordinates: automatic model builders. Chemical Reviews, 93(7):2567-2581, 1993.
J Andrew Grant, MA Gallardo, and Barry T Pickup. A fast method of molecular shape comparison: A simple application of a gaussian description of molecular shape. Journal of Computational Chemistry, 17(14): 1653-1666, 1996.
Emanuele Perola and Paul S Charifson. Conformational analysis of drug-like molecules bound to proteins: an extensive study of ligand reorganization upon binding. Journal of Medicinal Chemistry, 47(10):2499-2510, 2004.
Sereina Riniker and Gregory A. Landrum. Better informed distance geometry: Using what we know to improve conformation generation. Journal of Chemical Information and Modeling, 55 (12):2562-2574, 2015.
Victor Garcia Satorras, Emiel Hoogeboom, and Max Welling. E(n) equivariant graph neural networks. arXiv preprint arXiv: 2102.09844, 2021b.
Kristof T Schütt, Pieter-Jan Kindermans, Huziel E Sauceda, Stefan Chmiela, Alexandre Tkatchenko, and Klaus-Robert Muller. Schnet: A continuous-filter convolutional neural network for modeling” quantum interactions. arXiv preprint arXiv: 1706.08566, 2017.
Minkai Xu, Shitong Luo, Yoshua Bengio, Jian Peng, and Jian Tang. Learning neural generative dynamics for molecular conformation generation. arXiv preprint arXiv:2102.10240, 2021a.
Minkai Xu, Wujie Wang, Shitong Luo, Chence Shi, Yoshua Bengio, Rafael Gomez-Bombarelli, and Jian Tang. An end-to-end framework for molecular conformation generation via bilevel programming. arXiv preprint arXiv:2105.07246, 2021b.

1. A computer implemented method for determining a data representation of a molecule, the method comprising joint generation of a molecular graph and three-dimensional geometry for the molecule, the joint generation including:
determining a data representation of an initial partial molecule;
repeating incremental modification of the partial molecule to provide a generated molecule, in each repetition or at least some of the repetitions, incrementally adding an increment comprising one or more atoms to the partial molecule, and modifying the data representation for the partial molecule to include a molecular graph including the one or more atoms and the geometric information for said one or more atoms; and
providing a data representation of the partial molecule as a data representation of the generated molecule.

2. The method of claim 1, wherein incrementally adding the increment includes selecting the one or more atoms based on the partial molecule.

3. The method of claim 1, wherein incrementally adding the increment includes:
adding the one or more atoms to the molecular graph of the partial molecule; and
determining the geometric information for the one or more atoms added in the increment to the molecular graph.

4. The method of claim 1, further comprising providing the generated molecule for further physical or simulated evaluation of its chemical properties.

5. The method of claim 1, wherein at least one of (a) the incrementally adding of the increment comprising one or more atoms to the partial molecule, (b) the selecting of the one or more atoms based on the partial molecule, (c) the adding of the one or more atoms to the molecular graph of the partial molecule, and (d) the determining of the geometric information for the one or more atoms is performed using a machine learning model trained from a training set of molecules.

6. The method of claim 5, wherein the machine learning model comprises an artificial neural network.

7. The method of claim 5, wherein the training set of molecules is selected according to desired properties of the generated molecule.

8. The method of claim 5, further comprising training the machine learning model from the training set of molecules.

9. The method of claim 1, wherein incrementally adding the increment includes using a machine learning model adapted to preferentially generate molecules with a desired chemical property.

10. The method of claim 9, wherein the desired chemical property includes having a low-energy geometry.

11. The method of claim 1, wherein the initial partial molecule consists of a single atom.

12. The method of claim 1, wherein in at least some repetitions, a single atom is added in an increment.

13. The method of claim 12, wherein in each iteration only a single atom is added.

14. The method of claim 1, wherein each iteration further includes determining a label for each atom added in the increment, and determining bonding information between each atom added and one or more atoms of the partial molecule to which the increment is added.

15. The method of claim 14, wherein the label for an atom identifies the element of the atom.

16. The method of claim 14, wherein the bonding information includes at least one of an indication of whether or not a bond is present and a bond type between two atoms.

17. The method of claim 1, wherein the adding of geometric information includes adding location information for each atom added in the increment.

18. The method of claim 17, wherein adding the location information includes at least one of (a) determining physical distance information of an atom in the increment to two or more atoms in the partial molecule, (b) determining physical angle information of an atom in the increment to two or more atoms in the partial molecule, and (c) determining both the physical distance information and the physical angle information.

19. The method of claim 1, wherein the increment that is incrementally added depends at least in part on geometry of the partial molecule.

20. The method of claim 1, wherein the increment that is incrementally added is chosen randomly based on the partial molecule to which the increment is added.

21. The method claim 20, wherein multiple molecules are formed with each molecule being randomly formed from a same initial partial molecule randomly choosing different increments in the repeated incremental modification.

22. The method of claim 21, wherein randomly forming a molecule includes determining a distribution over possible increments for addition to the molecular graph, and selecting a particular increment using the distribution.

23. The method of claim 14, wherein determining the label for an atom added in the increment includes using a first artificial neural network that takes as input a representation of at least one of (a) a representation of the molecular graph of the partial molecule, (b) a representation of the three-dimensional geometry of the partial molecule, and (c) representations of both the molecular graph and the three-dimensional geometry of the partial molecule.

24. The method of claim 23, wherein the output of the first artificial neural network includes a distribution of possible labels of the atom that is added.

25. The method of claim 14, wherein determining the bonding information for an atom added in the increment includes using a second artificial neural network that takes as input a representation of at least one of (a) a representation of the molecular graph of the partial molecule, (b) a representation of the three-dimensional geometry of the partial molecule, and (c) a representation of the label or distribution of labels for an atom added.

26. The method of claim 18, wherein determining physical distance information of an atom in the increment to one or more atoms in the partial molecule includes using a third artificial neural network that takes as input at least (a) a representation of the three-dimensional geometry of the partial molecule, (b) a representation of a label or a distribution of labels of the atom to be added, and (c) a representation of the molecular graph of the partial molecule.

27. The method of claim 26, wherein the third artificial neural network is used repeatedly to determine physical distance information to different atoms of the partial molecule.

28. The method of claim 18, wherein determining the physical angle information of an atom in the increment to two or more atoms in the partial molecule includes using a fourth artificial neural network that takes as input at least (a) a representation of the three-dimensional geometry of the partial molecule, (b) a representation of a label or a distribution of labels of the atom to be added, and (c) a representation of the molecular graph of the partial molecule.

29. The method of claim 28, wherein one or more of the neural networks are trained using a molecular graph and three-dimensional geometry information for a database of valid molecules.

30. The method of claim 28, wherein one or more of the first through fourth neural networks are trained using a molecular graph and three-dimensional geometry information for a database of molecules having a desired chemical property.

31. The method of claim 28, wherein one or more of the neural networks are adapted using molecular graph and three-dimensional geometry information for a database of molecules having a desired chemical property after training the neural networks using a database of molecules that do not necessarily have the desired chemical property.

32. A non-transitory machine-readable medium comprising instructions stored thereon, said instructions when executed using a computer processor cause said processor to determine a data representation of a molecule the determining comprising joint generation of a molecular graph and three-dimensional geometry for the molecule, the joint generation including:
determining a data representation of an initial partial molecule;
repeating incremental modification of the partial molecule to provide a generated molecule, in each repetition or at least some of the repetitions, incrementally adding an increment comprising one or more atoms to the partial molecule, and modifying the data representation for the partial molecule to include a molecular graph including the one or more atoms and the geometric information for said one or more atoms; and
providing a data representation of the partial molecule as a data representation of the generated molecule.

33. (canceled)

34. A data processing system comprising means for carrying out determining a data representation of a molecule the determining comprising joint generation of a molecular graph and three-dimensional geometry for the molecule, the joint generation including:
determining a data representation of an initial partial molecule;
repeating incremental modification of the partial molecule to provide a generated molecule, in each repetition or at least some of the repetitions, incrementally adding an increment comprising one or more atoms to the partial molecule, and modifying the data representation for the partial molecule to include a molecular graph including the one or more atoms and the geometric information for said one or more atoms; and
providing a data representation of the partial molecule as a data representation of the generated molecule.

35. (canceled)

36. The method of claim 3, wherein
the method further comprises providing the generated molecule for further physical or simulated evaluation of its chemical properties;
wherein at least one of (a) the incrementally adding of the increment comprising one or more atoms to the partial molecule, (b) the selecting of the one or more atoms based on the partial molecule, (c) the adding of the one or more atoms to the molecular graph of the partial molecule, and (d) the determining of the geometric information for the one or more atoms is performed using a machine learning model trained from a training set of molecules;
wherein the machine learning model comprises an artificial neural network;
wherein the training set of molecules is selected according to desired properties of the generated molecule;
the method further comprises training the machine learning model from the training set of molecules;
wherein each iteration further includes determining a label for each atom added in the increment, and determining bonding information between each atom added and one or more atoms of the partial molecule to which the increment is added;
wherein the label for an atom identifies the element of the atom;
wherein the bonding information includes at least one of an indication of whether or not a bond is present and a bond type between two atoms;
wherein the adding of geometric information includes adding location information for each atom added in the increment;
wherein adding the location information includes at least one of (a) determining physical distance information of an atom in the increment to two or more atoms in the partial molecule, (b) determining physical angle information of an atom in the increment to two or more atoms in the partial molecule, and (c) determining both the physical distance information and the physical angle information; and
wherein determining the label for an atom added in the increment includes using a first artificial neural network that takes as input a representation of at least one of (a) a representation of the molecular graph of the partial molecule, (b) a representation of the three-dimensional geometry of the partial molecule, and (c) representations of both the molecular graph and the three-dimensional geometry of the partial molecule;
wherein determining the bonding information for an atom added in the increment includes using a second artificial neural network that takes as input a representation of at least one of (a) a representation of the molecular graph of the partial molecule, (b) a representation of the three-dimensional geometry of the partial molecule, and (c) a representation of the label or distribution of labels for an atom added;
wherein determining physical distance information of an atom in the increment to one or more atoms in the partial molecule includes using a third artificial neural network that takes as input at least (a) a representation of the three-dimensional geometry of the partial molecule, (b) a representation of a label or a distribution of labels of the atom to be added, and (c) a representation of the molecular graph of the partial molecule;
wherein determining the physical angle information of an atom in the increment to two or more atoms in the partial molecule includes using a fourth artificial neural network that takes as input at least (a) a representation of the three-dimensional geometry of the partial molecule, (b) a representation of a label or a distribution of labels of the atom to be added, and (c) a representation of the molecular graph of the partial molecule; and
wherein one or more of the first through fourth neural networks are trained using a molecular graph and three-dimensional geometry information for a database of molecules having a desired chemical property.
